(19) United States
(12) Patent Application Publication
(10) Pub. No.: US 2005/0118711 A1
(43) Pub. Date: Jun. 2, 2005

(54) Device for applying liquid media and corresponding method

(76) Inventors: Alfred Nordheim, Tuebingen (DE); Winfried Kammer, Tuebingen (DE)

Correspondence Address: NATH & ASSOCIATES, 1030 15th STREET, NW, 6TH FLOOR, WASHINGTON, DC, 20005, US

(21) Appl. No.: 10/506,876

(22) PCT Filed: Mar. 5, 2003
(86) PCT No.: PCT/EP03/02209

(30) Foreign Application Priority Data
Mar. 5, 2002 (DE) 10210908.7

(51) Int. Cl. C12N 5/08; C12M 1/34

(52) U.S. Cl. 435/366; 435/287.1

(57) ABSTRACT

The invention relates to a device (31) for applying liquid media, particularly culture media and/or reaction media, and a method fur generating suitable reaction and/or cultivation conditions. According to one preferred embodiment, said device stands on four legs (32). The drops (33) can be seen on the bottom side of the device (31).

The invention relates to a device for applying liquid media, particularly culture media and/or reaction media, and to a method for generating suitable reaction and/or cultivation conditions.

In modern medical science and biotechnology and in related fields, there is a great demand for devices with which it is possible to test substances or substance combinations quickly, efficiently and, if possible, with little preparative work, for their embryotoxic properties, toxicity (toxicity profile), efficacy, effective concentration, etc. Thus, for example, in chemotherapy, it is often first of all determined whether the chemostatics can kill the tumors. This is in most cases tested in the cell culture. Here, tumor cells are taken from the patient and cultivated. After a sufficient yield of tumor cells has been obtained, these cells are then exposed to various chemostatics. Those chemostatics which achieve the best results in the cell culture, i.e. which have killed the most cancer cells at the lowest concentration, are then employed in the subsequent chemotherapy. However, it often happens that the results achieved in vitro cannot be reproduced in vivo. This is due, among other things, to the fact that the conditions which prevail in an organism cannot be readily imitated in a cell culture.

Difficulties also arise in testing substances for their embryotoxic properties. A standard method for carrying out such tests is the incubation of what are called embryoid bodies. Embryoid bodies develop from embryonic stem cells through cell aggregation and subsequent cell differentiation. From the cells of embryoid bodies, it is possible, by means of incubation under suitable cultivation conditions, to raise cells of defined differentiation status (e.g. neuronal cells, myocytes or blood stem cells). To date, it has not been possible to raise embryoid bodies in a form in which they a) can be sown reliably (homogeneously and with possible automation), b) can be safely handled (robotics) in volumes of, for example, 40 μl and over, and c) can be analyzed with high parallelization (automation). Thus, only individually raised embryoid bodies have hitherto been available for this standard method. The problems arising in the cultivation of embryoid bodies are due, inter alia, to the fact that embryonic stem cells aggregate to form embryoid bodies only under certain conditions. Decisive factors are, for example, the concentration of embryonic stem cells and the supply of suitable nutrients. In addition, the cells must not be given any opportunity to adhere to a surface. Since individually cultivated embryoid bodies for testing for embryotoxic properties can be made available only at considerable expense and with considerable preparatory work, and since the cultivation and reaction conditions vary enormously in each test, the results obtained cannot be reliably compared with one another.

A number of companies have therefore produced devices promising a solution to these problems. For example, the company MWG AG has marketed what is called an Affymetrix Arrayer, which is intended to be used for automated production of DNA arrays. This Affymetrix Arrayer makes use of what is called Pin-and-Ring™ technology. In this technology, a so-called pin (metal spike) is passed through a liquid membrane which is held by a ring. This is similar to the ring blown through to produce soap bubbles. As soon as the pin has been pushed through the liquid membrane, a defined amount of liquid can then be loaded onto the array with this pin. This system, however, can only be used to transfer small amounts of liquid.

Another company, Berkeley Lab. Inc., produces so-called microcrystallization robots. These devices are used in particular when the three-dimensional structure of proteins is to be investigated by crystallography technology. To understand the function of a protein, its tertiary structure must be elucidated. To do this, crystal structure analysis is often used. In crystal structure analysis, the spatial arrangement of the atoms in crystalline solids is determined with the aid of X-ray, electron or neutron beams whose wavelengths correspond approximately to the atomic distances in crystal lattices. In the determination of the tertiary structure of proteins, this technique is referred to as protein X-ray crystallography. In the production of the crystals, however, differing environmental conditions often result in a lack of uniformity of structure, which is referred to as a crystal defect and can lead to false results in the crystal structure analysis.

Microtiter plates represent another possibility for performing a large number of tests simultaneously. In the case of microtliter plates, the (liquid) media and also the substances to be tested are introduced directly into so-called cavities, which basically correspond to reaction chambers, with a base and side boundaries. The actual reaction/cultivation then takes place in these chambers.

Further devices for applying media are available on the market or are being tested in research departments and at universities. All of them, however, have various disadvantages. Thus, in the already described method for testing substances for their embryotoxic properties, one problem is that the embryonic stem cells do not aggregate into homogeneous embryoid bodies in a controlled and reproducible manner. When they are plated out, for example, on a microtiter plate, the stem cells grow across the entire bottom surface of the microtiter plate, as a result of which aggregation to embryoid bodies does not take place. Also, the devices already described are not able to afford more suitable cultivation/reaction conditions. Attempts are therefore made, on a small scale, to create a so-called hanging drop by applying the liquid (in the case of stem cells the cultivation medium) to a slide, for example, which is then rotated.

These “hanging drops” have a number of advantages. Thus, on the one hand, the tested substances are completely enclosed by the liquid, which permits sufficient supply of the necessary factors, for example ions, salts, differentiation factors, toxins, etc. On the other hand, “hanging drops” permit the aggregation of embryonic stem cells to embryoid bodies, because the cells in the drop sink downward but do not make any contact there with a solid surface. For lack of other sites on which to adhere, the embryonic stem cells aggregate and form embryoid bodies. The surface tension of the drops prevents escape of embryonic stem cells and embryoid bodies from the drops. However, transferring this principle to a larger scale for the production of suitable cultivation/reaction conditions for standardized tests, as are needed for example in the evaluation of pharmaceuticals, has hitherto been unsuccessful, since no solutions have been found to the problems relating to parallel handling of these “hanging drops”. Thus, when applying several drops to a surface, there is always the risk of drops running into one another. Moreover, only relatively small volumes can be applied, since, when a critical volume is exceeded, drops become uncontrollable when the support is moved. With a drop volume of just 20 μl, drops can move significantly on hydrophobic bases during handling. To automate the production of homogeneous hanging drops, one simple and reproducible possibility is that of so-called top-loading. In the latter, a defined volume of liquid is applied to a base from above. When the base is turned round, the drop hangs downward. When it is turned round, the drops, depending on their size, can leave the site of their application. It is generally a rule, however, that the larger the drop, the more uncontrollable it is to handle. On the other hand, larger drops permit a longer cultivation period without exchange of medium (this leads, for example, to longer-lasting differentiation processes), a greater cell quantity can be applied, etc. In addition, in larger drops, evaporation processes do not have such a marked effect as in smaller drops, because the surface-to-volume ratio is considerably more favorable than in smaller drops. The reproducibility of the ongoing processes in the drop is thus significantly enhanced.

It follows from this that the drops acting as actual reaction vessels
should be as large as possible,
should be able to be applied in a reproducible manner,
should be produced and used with a high degree of parallelization/automation.

A device and a method for applying liquid media are therefore needed. In the context of the above statements, the volumes of media that can be applied should on the one hand be large enough to allow long-lasting and complex (bio)chemical and/or biological reactions to take place. On the other hand, the results should be reproducible.

It is therefore an object of the invention to make available a device and a method which can be used universally to produce such suitable conditions and which can help overcome the described disadvantages of the prior art. Another aim of this invention is to make available a device and a method which permit production of a multiplicity of (identical) cultivation/reaction conditions with comparatively little preparatory work.

This object is achieved by the device and the method having the features of independent claims 1, 25, 27 and 35. Preferred embodiments are set out in the dependent claims. The wording of all the claims is incorporated by reference in the content of this description.

It has surprisingly been found that stabilization of liquid media is achieved by provision of sharp-edged boundaries, so that, even in the case of larger volumes, “hanging drops” are created by rotation, and the drops do not tear off from the surface. In addition, a displacement of the drops can also be avoided in this way.

The subject of the invention is therefore a device for applying liquid media, particularly culture media and/or reaction media, the device having at least one substantially planar elevation made of a hydrophobic material/support material, and this planar elevation having at least one, in particular two, sharp-edged boundaries, in particular edges, arranged parallel to one another. Planar elevation is here intended to signify any elevation with a substantially plane top limit surface. The elevation is preferably cube-shaped or block-shaped. Truncated pyramid-shaped or similar elevations are also possible. On its top limit surface, the elevation can also have a slightly convex or concave design. The important feature is that the liquid media applied to the elevation made of a hydrophobic support material are fixed as drops by the sharp-edged boundary(boundaries) of the top limit surface. According to the invention, it is furthermore proposed to change round the corresponding principle of action. Thus, a hydrophilic support material can be used, on which a hydrophobic liquid medium is then applied. The fixing likewise takes place via the sharp-edged boundary (boundaries) of the top limit surface.

According to a preferred embodiment, the dimension of a planar elevation in the longitudinal direction is between about 2 and about 7 mm, preferably between about 3 and about 5 mm. In another preferred embodiment, the dimension of a planar elevation in the transverse direction is between about 3 and about 9 mm, preferably between about 4 and about 7 mm. In one illustrative embodiment, the elevations are 5×7 mm in size, and in another example they are about 3×4 mm in size. Other dimensions of the elevation are also claimed according to the invention insofar as the elevation has at least one sharp-edged boundary for stabilizing the liquid medium. If the dimensions of the elevations are relatively small, this can have the advantageous effect that in this way the drop volume is also made smaller, and the drops thereby gain in stability.

In a particularly preferred embodiment of the device according to the invention, the planar elevation is designed as a narrow, elongate elevation. A band of liquid can advantageously be applied to an elongate elevation of this kind. In the field of tissue engineering for example, elongate products such as vessels, in particular blood vessels, or fibers, in particular nerve fibers or muscle fibers, can be cultivated in this liquid band. The dimensions of these elongate elevations depend of course on the particular case of use. The length can range from a few millimeters, for example about 2 or about 3 mm, to the entire length of the device according to the invention, that is to say several centimeters, for example up to 13 cm. The width likewise depends on the case of use and can, for example, be between about 1 mm and about 5 mm.

In a further preferred embodiment, the planar elevation is between 1 and 5 mm high, preferably ca. 2 mm high. The height of the elevation is adapted in particular to the hydrophobic support material used and to the relevant method for production of suitable devices. Thus, in the case of milling for example, the height of an elevation depends on the milling depth and the line width, both of which can vary depending on the design of the device.

In a further preferred embodiment of the invention, the device has at least two, preferably a multiplicity of, planar elevations. The number of planar elevations can be at least 18 in the longitudinal direction and at least 9 in the transverse direction. In another preferred embodiment, the number of planar elevations is 12 in the longitudinal direction and 8 in the transverse direction. In terms of the arrangement and number of the elevations, this embodiment corresponds substantially to a standard microtiter plate with 96 wells, which may be advantageous for many uses. The number of elevations depends on the overall dimensions of the device, on the intended use (e.g. cell culture), etc. If the device has a plurality of planar elevations, these elevations, in another preferred embodiment, are located between 1 and 4 mm, preferably ca. 2 mm, from one another. Preferred distances of the elevations from one another also lie between 0.5 and 1.5 mm.

In a preferred embodiment, the sharp-edged boundary is from a right angle to an acute angle. According to the invention, acute angle is to be understood as an angle of <90°.

In principle, a wide variety of materials/support materials can be used in the invention. These are in particular plastics, for example organic polymers.

In a particularly preferred embodiment, the device consists at least partially, preferably completely, of at least one transparent material/support material. For this purpose, possible materials are polystyrene and/or Plexiglas. The use of a transparent support material has the advantage that evaluation with the aid of suitable visual methods, e.g. microscopy, fluorescence measurement, etc., can take place directly on the device itself.

In a further preferred embodiment, the device has an overall height of between 10 and 30 mm, preferably between 10 and 23 mm. The dimensions of the device can be between 100 and 150 mm, preferably ca. 130 mm, in the longitudinal direction, and between 80 and 100 mm, preferably ca. 90 mm, in the transverse direction, as is claimed in a further preferred embodiment. The dimensions of the device can be adapted to the desired site of use. Thus, for example, when used in a cell incubator, the device will be correspondingly adapted to the dimensions of the interior of the latter. If the device according to the invention is to be fitted into a round vessel, for example, the maximum longitudinal and transverse dimensions of the device then correspond to the diameter of the round vessel.

For the purpose of gripping it, the device, in a further preferred embodiment, has at least one way and preferably two ways of being held, so that handling and transporting of the device are made easier. The possible holds are preferably afforded in particular by two grips which are arranged in particular on opposite outer edges of the device. These can, for example, be designed as simple projections on the edges.

In a further preferred embodiment of the device according to the invention, said device is designed in such a way that it can be placed on a base, in particular on a supporting frame. In this case, provision is preferably made that the device can be placed in both orientations so that, when it is placed, the side to be loaded with samples can point upward, which makes loading very straightforward. In the other case, the surface provided for loading then points downward, so that this for example would be the position for cultivation or reaction in the hanging drop. For this purpose, lateral projections can advantageously be provided on two outside edges of the device, with which lateral projections the device can on the one hand be transported and can on the other hand be placed on a suitable base/frame in the respective desired orientation.

In a further preferred embodiment of the device according to the invention, it has at least one support, preferably several supports, in particular legs. If, for example, the device according to the invention is used on its own, then the device can have four legs in order to give it a stable position.

In a particularly preferred embodiment, at least two devices can be releasably secured to one another or on one another. Thus, for example, according to the invention, it is possible for several devices to be interconnected by lock-type, plug-type or clamp-type couplings. The devices in this case can be arranged alongside one another and/or over one another (stacked on top of one another). These interconnected and/or stacked devices can then, for example, be transported and/or incubated together, e.g. in the cell culture. Alternatively, insert devices for one to several devices can be used for transport and incubation.

In further preferred embodiments of the invention, between 10 and 80 μl, preferably 40 to 80 μl, of liquid medium can be applied per elevation.

The device according to the invention can be turned at least 90°, preferably ca. 180°, after application of the liquid media, as is claimed in a further embodiment. By turning the device, so-called “hanging drops” are then created.

A corresponding frame on which to place the described device is also covered by the invention. This frame is preferably designed such that two to four spars, abutting one another approximately at right angles and secured to one another, form a frame which serves as a base and onto which the device according to the invention, having projections on at least two edges, is placed. On the upper edges of the supporting frame thus formed, recesses can be provided at a suitable location in order to ensure a secure hold for the projections of the device to be placed thereon. The projections on the device to be placed thereon, which can advantageously also be used as grips, and the corresponding recesses in the supporting frame, are preferably situated on opposite sides of the device to be placed thereon and of the supporting frame, respectively. In a particularly preferred embodiment, the supporting frame has three spars which, as it were, form an open rectangle. However, it may also be preferable for the supporting frame to consist of two spars abutting one another approximately at right angles and secured to one another. It may also be preferable for the supporting frame to be formed by four spars which, as it were, form a closed rectangle.

A method for generating suitable reaction and/or cultivation conditions after application of liquid media is also claimed, which method uses a device according to the invention. The cultivation conditions can be suitable conditions for proliferation and/or differentiation assays for eukaryotic cells. The eukaryotic cells are in particular human or animal cells, or cell formations, tissues, organoids or organs, as is claimed in a further preferred embodiment.

The method according to the invention can further be characterized in that the cultivation conditions are growth and/or differentiation conditions for eukaryotic cells for the purpose of tissue culture and in particular for the purpose of tissue engineering. Moreover, the method can advantageously be used such that the cultivation conditions are stimulating and/or inhibiting and/or destroying conditions for the cultivation of tumor cells and/or tumor tissues. With the aid of such a method, it is possible, for example, to test reagents that are to be used in chemotherapy. In this artificial system, it is possible to test, prior to the actual therapy itself, whether certain chemotherapeutic agents for certain tumor cells and/or tissues have the desired effect or not.

The method according to the invention can also be such that the cultivation conditions are growth and/or differentiation conditions for cell aggregates and/or tissues for influencing angiogenic processes in these cell aggregates and/or tissues. In this way, for example, it would be possible to investigate and test different factors which stimulate or inhibit such angiogenic processes which may also be implicated in the development of tumors.

According to a further preferred embodiment of the method, the human or animal cells are stem cells, in particular embryonic stem cells. Embryonic stem cells are so-called totipotent cells, i.e. they can differentiate into all cell types. In a preferred embodiment, these embryonic stem cells, after application, aggregate and/or differentiate to form embryoid bodies.

In a further preferred embodiment, the reaction conditions can be suitable crystallization and/or X-ray structure analysis conditions. The device according to the invention can be used, for example, to generate crystallization chambers in drop form. After crystallization of the proteins, the three-dimensional structure of the proteins can be established, for example, with the aid of X-ray, electron or neutron beams.

The invention also covers a method for using drops as a reaction site, in which method a liquid medium, for example a culture medium for the cell culture, is applied to (loaded onto) an above-described device, and the device is then oriented with the loaded side facing downward. The underside can in this case be substantially horizontal or also at an angle of, for example, 90° or less with respect to the base. The reaction site thus formed can be used, for example, for the cultivation of biological material, in particular eukaryotic cells, cell aggregates and/or cell tissues. On the other hand, it is also advantageously possible to use such reaction sites for crystallization processes with a view to structure analyses. Moreover, the invention of course also covers other possible applications in which a drop can be used as reaction site.

In a preferred embodiment, in order to remove the drops from the device according to the invention, the device, with the loaded side facing downward, can be brought toward a substantially planar surface in such a way that the drops settle on this surface. To do this, the device and the surface are brought close enough together until the drops touch the surface and thus pass onto the surface. Suitable surfaces for this aspect of the invention can for example consist at least partially of glass and/or plastic.

In a further preferred embodiment of this aspect of the invention, the drops are removed with the aid of at least one spacer located on the surface and/or on the device with the drops. This avoids a situation where the samples in the drops, for example cells or tissues, are damaged by crushing.

In another preferred embodiment of this method, the device for removing the drops, with the loaded side facing downward, is brought toward at least one depression so that the drops come into contact with at least one side wall of the depression and run down it. Suitable depressions are, advantageously, those in a microtiter plate or the like.

Further details and features of the invention will become clear from the following description of preferred embodiments of the device according to the invention and of the method, in conjunction with the claims. The respective features may be realized individually or in combination with one another.

In the figures:

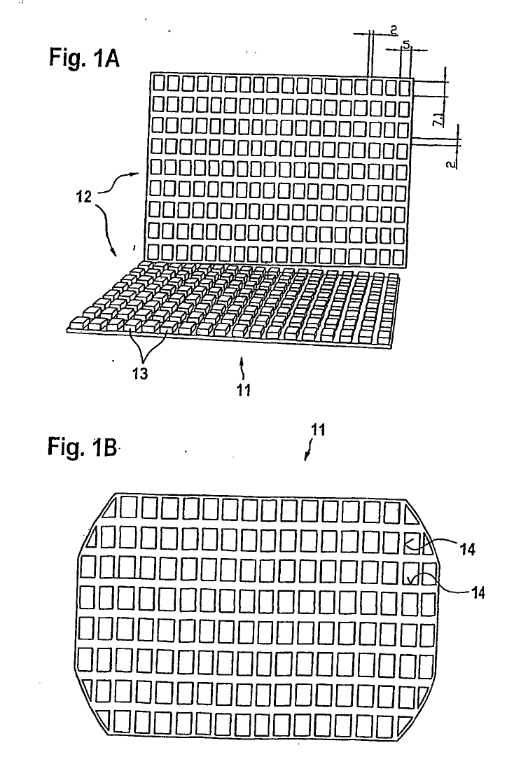

FIG. 1 shows a plan view and side view of a possible embodiment of the device according to the invention.

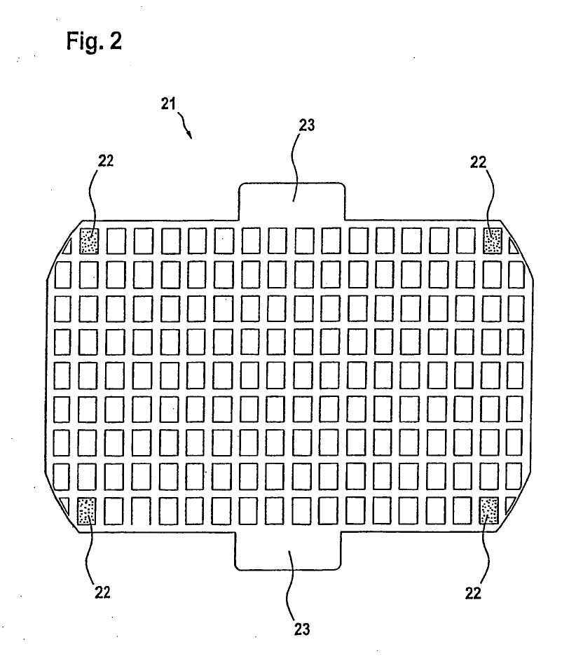

FIG. 2 shows a plan view of a device according to the invention in the unloaded state.

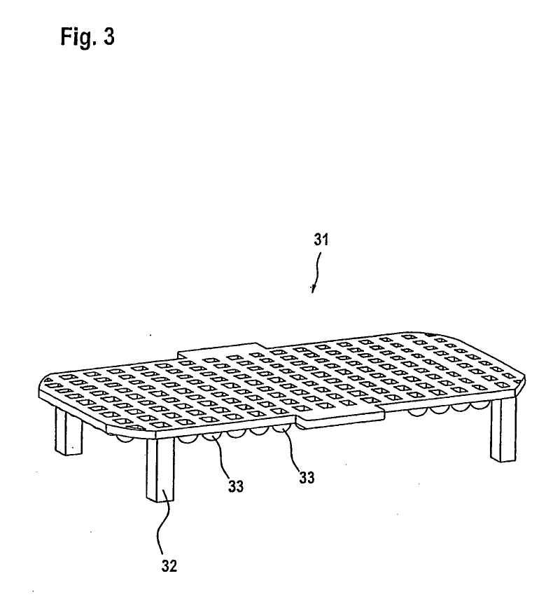

FIG. 3 shows a side view of a device according to the invention loaded with liquid.

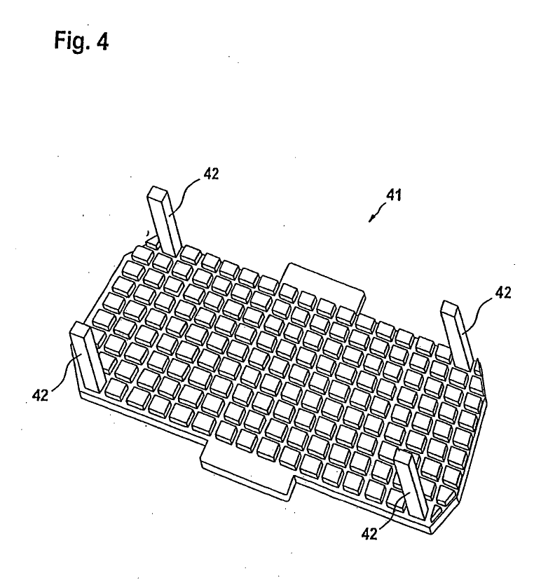

FIG. 4 shows a plan view of an unloaded device according to the invention standing on its side.

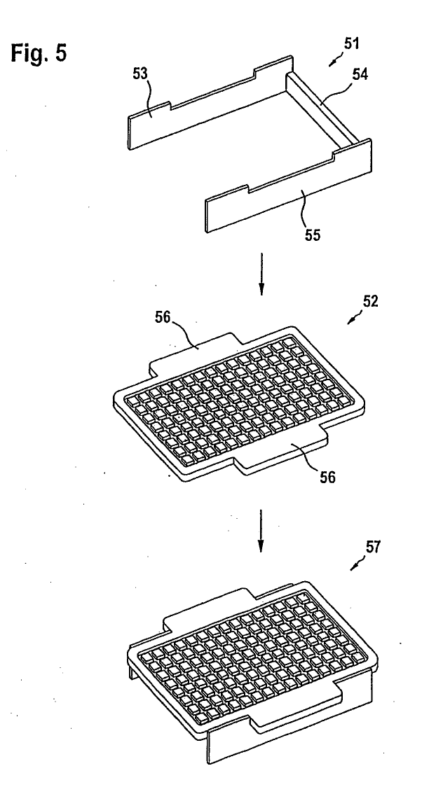

FIG. 5 shows an oblique view of a supporting frame according to the invention and of a device according to the invention, separately and in the assembled state.

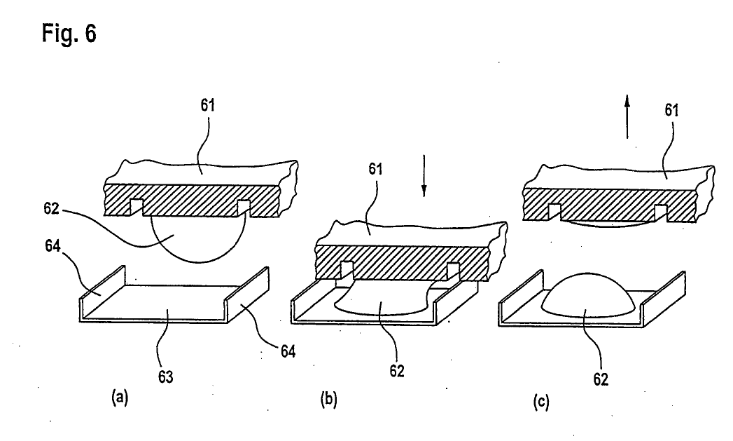

FIG. 6 shows a preferred embodiment according to the invention for removing the drops.

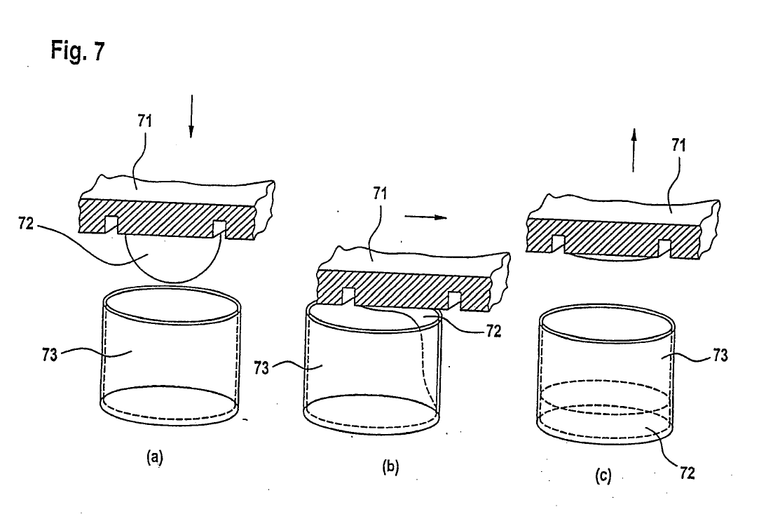

FIG. 7 shows a further preferred embodiment according to the invention for removing the drops.

FIG. 1 is a diagrammatic representation of a possible embodiment of the device (11) according to the invention. The device (11) consists of a support material (12) and of a multiplicity of elevations (13) with a substantially planar top limit surface. Moreover, the sharp-edged boundaries (14) are also shown. The individual (planar) elevations (13) are separated from one another by in each case ca. 2 mm. The dimensions of an elevation are ca. 5 mm in the longitudinal direction and ca. 7.1 mm in the transverse direction. In total, 18 elevations are shown in the longitudinal direction, and 9 in the transverse direction.

FIG. 1A shows a plan view and perspective side view of the device 11. Such a device can, for example, be made from a polystyrene plate (ca. 4 mm×130 mm×130 mm) or a Plexiglas block. A milling tool is used to mill out the elevations depicted, in which process linear depressions of ca. 2 mm in width and ca. 2 mm in depth are milled out from the plate or block. The device can then be worked, depending on the dimensions it is intended to have. For example, protuberances on the polystyrene plate can serve as grips. Also, for example, the corners of the device (11) can be rounded, as is shown in FIG. 1B, so that it can be inserted into a round vessel of corresponding diameter. Moreover, legs can also be attached to the device, for example by adhesive bonding, so that the device has better stability.

FIG. 2 shows a plan view of a device according to the invention. This device was produced in the way described for FIG. 1. The device 21 designed as a plate stands on four legs 22 and is still in the unloaded state. Arranged on both long sides of the device 21 are projections 23 which can be used for transporting the device and placing it on a suitable frame. The plate is loaded by means of a certain volume of a suitable solution, for example of a culture medium, being applied to each elevation, which in this case is on the side facing away from the viewer. For loading the device according to the invention, said device advantageously lies on its back, i.e. legs 22 would point vertically upward. The applied drops take on the substantially rectangular shape, rounded at the corners, even during loading. Once the loaded plate has been placed on its legs, rounded, clear demarcations of the drop-shaped solution adhering to each elevation can be seen at the margins (edges) of each elevation, on the side facing away from the viewer. The drops would be hanging down in the viewing direction. Each single one of these drops is in this case stabilized by the sharp-edged boundaries or edges at the margins of each elevation, so that pulling-off or displacement of the drop is prevented. This is shown in FIG. 3.

FIG. 3 shows a device 31 loaded with culture medium and set in the position of use. Here too, the device stands on four legs 32. The view shown is an oblique view from the side. The drops 33 can be discerned on the underside of the device 31.

FIG. 4 shows a device 41 standing on its side, at an angle. The legs 42 of the plate are approximately horizontal to the base, while the plate is substantially vertical to the base. The device is shown in the unloaded state. This position can also be used at suitable inclination for cultivation.

FIG. 5 shows at the top a supporting frame 51 which is suitable for placement of a device 52 according to the invention, as is shown in the middle. The supporting frame 51 consists of three spars 53, 54, 55 which abut one another at right angles to the narrow faces and which form a rectangular frame. Arranged on the upper edges of the two opposite spars 53 and 55, there is in each case a recess which is provided for receiving projections 56 on two opposite edges of the device 52. Moreover, the upper edge of the face of the spar 54 is slightly lower than the upper edges of the side spars 53 and 55, with the result that the drops on the device 52 cannot come into contact with the end spar 54 of the frame 51. This therefore ensures a stable and secure hold of the device 52 according to the invention on the frame 51, as can be seen from the bottom part of FIG. 5. The device 52 according to the invention can be placed on the supporting frame 51 either with the surface to be loaded, or the already loaded surface, pointing upward or downward. Thus, the supporting frame 51 can be used for loading or working of the samples and also, for example, for cultivation in the hanging drops.

FIG. 6 shows the removal of the drops from a device according to the invention. The device 61 according to the invention with the drop 62 hanging from it is (a) brought up to a substantially planar surface 63 until (b) the drop 62 touches the surface 63 and can be deposited on the surface 63. After the drop 62 has been deposited on the surface 63, the device 61 is removed again (c). FIG. 6 also shows two spacers 64 on the surface 63, these spacers 64 ensuring that a minimum distance is maintained between the device 61 and the substantially planar surface 63, so as to rule out crushing of the product in the drop 62.

FIG. 7 shows a further advantageous embodiment for removal of the drop. Here, the drop 72 located on the device 71 according to the invention is (a) brought toward a depression 73. The device 71 is then moved so that (b) the drop 72 touches a side wall of the depression 73 and runs down this side wall. Finally, the device 71 is removed from the depression 73. The drop 72 is now located (c) in the depression 73. The depression 73 is advantageously the cavity of a microtiter plate or similar. Of course, the depression can also be a separate reaction vessel.

1. A device for applying liquid media, particularly culture media and/or reaction media, characterized in that the device has at least one elevation made of a hydrophobic material and with a substantially planar top limit surface, and this planar elevation has, on the top planar limit surface, at least one, in particular at least two, sharp-edged boundaries, in particular edges, arranged parallel to one another.

2. The device as claimed in claim 1, characterized in that the dimension of a planar elevation in a first direction, preferably in the longitudinal direction, is between about 2 and about 7 mm, preferably between about 3 and about 5 mm.

3. The device as claimed in claim 1, characterized in that the dimension of a planar elevation in a first direction, preferably in the longitudinal direction, is between 4 and 7 mm, preferably ca. 5 mm.

4. The device as claimed in claim 1, characterized in that the dimension of a planar elevation in a second direction, preferably in the transverse direction, is between about 3 and about 9 mm, preferably between about 4 and about 7 mm.

5. The device as claimed in claim 1, characterized in that the dimension of a planar elevation in a second direction, preferably in the transverse direction, is between 5 and 9 mm, preferably ca. 7 mm.

6. The device as claimed in claim 1, characterized in that the planar elevation is a narrow, elongate elevation.

7. The device as claimed in claim 1, characterized in that the planar elevation is between 1 and 5 mm high, preferably ca. 2 mm high.

8. The device as claimed in claim 1 characterized in that the device has at least two, preferably a multiplicity of, planar elevations.

9. The device as claimed in claim 8, characterized in that the number of planar elevations is at least 18 in the longitudinal direction and at least 9 in the transverse direction.

10. The device as claimed in claim 8, characterized in that the number of planar elevations is 12 in the longitudinal direction and 8 in the transverse direction.

11. The device as claimed in claim 8, characterized in that the distance between two adjacent planar elevations is between 0.5 and 4 mm, preferably between 1 and 2 mm, in particular ca. 2 mm.

12. The device as claimed in claim 1, characterized in that the sharp-edged boundary is designed at an acute angle.

13. The device as claimed in claim 1, characterized in that it has a preferably rectangular main body on which the planar elevations are formed.

14. The device as claimed in claim 1, characterized in that the device consists at least partially, preferably completely, of at least one transparent material.

15. The device as claimed in claim 14, characterized in that the material is polystyrene and/or Plexiglas.

16. The device as claimed in claim 1, characterized in that the dimensions of the device are between 100 and 150 mm, preferably ca. 130 mm, in a first direction, preferably in the longitudinal direction, and between 80 and 110 mm, preferably ca. 90 mm, in a second direction, preferably in the transverse direction.

17. The device as claimed in claim 1, characterized in that the device has an overall height of between 10 and 30 mm, preferably between 10 and 23 mm.

18. The device as claimed in claim 1, characterized in that the device has at least one grip, preferably two grips.

19. The device as claimed in claim 1, characterized in that the device is designed so that it can be placed on a supporting frame, the device preferably being able to be placed thereon with the surface to be loaded, or the loaded surface, facing upward or downward.

20. The device as claimed in claim 1, characterized in that the device has at least one support, preferably several supports, in particular legs.

21. The device as claimed in claim 1, characterized in that it is designed to be stackable.

22. The device as claimed in claim 21, characterized in that at least two devices can be releasably secured on one another.

23. The device as claimed in one of the preceding claims, claim 1 characterized in that between 10 and 80 μl, preferably 40 to 50 μl, of liquid medium can be applied per elevation.

24. The device as claimed in claim 1, characterized in that the device can be turned at least 90°, preferably ca. 180°, after application of the liquid media.

25. A device for holding the device as claimed in claim 1, characterized in that the device consists of 2 to 4 spars, in particular 3 spars, which form an approximately rectangular frame.

26. The device as claimed in claim 25, characterized in that, on the upper edges, it has at least one recess, preferably at least two recesses, which are provided for receiving corresponding projections on the device to be held on it.

27. A method for generating suitable reaction and/or cultivation conditions after application of liquid media, characterized by the use of a device having at least one feature of claim 1.

28. The method as claimed in claim 27, characterized in that the cultivation conditions are proliferation and/or differentiation conditions for eukaryotic cells, in particular human or animal cells.

29. The method as claimed in claim 27, characterized in that the method is used for the purpose of tissue culture and/or tissue engineering.

30. The method as claimed in claim 27, characterized in that the cultivation conditions are stimulating and/or inhibiting and/or destroying conditions for the cultivation of tumor cells and/or tumor tissues.

31. The method as claimed in claim 27, characterized in that the cultivation conditions are growth and/or differentiation conditions for cell aggregates and/or tissues for influencing angiogenic processes in these cell aggregates and/or tissues.

32. The method as claimed in claim 28, characterized in that the human or animal cells are stem cells, in particular embryonic stem cells.

33. The method as claimed in claim 32, characterized in that the embryonic stem cells, after application to the device, aggregate and/or differentiate to form embryoid bodies.

34. The method as claimed in claim 27, characterized in that the reaction conditions are crystallization and/or X-ray structure analysis conditions.

35. A method using drops as a reaction site, characterized in that a liquid medium is applied to the device as claimed in claim 1, and the device, with the loaded side facing downward, is oriented substantially horizontally or at an angle of about 90° or less to a base.

36. The method as claimed in claim 35, characterized in that, to remove the drop, the device, with the loaded side facing downward, is brought toward a substantially planar surface in such a way that the drop settles on the surface.

37. The method as claimed in claim 36, characterized in that the surface is provided with at least one spacer.

38. The method as claimed in claim 35, characterized in that, to remove the drop, the device, with the loaded side facing downward, is brought toward at least one depression so that the drop touches at least one side wall of the depression and runs down it.

39. The method as claimed in claim 38, characterized in that the depression is a depression of a microtiter plate.
